(19) United States
(12) Patent Application Publication
(10) Pub. No.: US 2020/0316359 A1
(43) Pub. Date: Oct. 8, 2020

(54) MULTI-USE BLOOD CONTROL CATHETER ASSEMBLY

(71) Applicant: Becton, Dickinson and Company, Franklin Lakes, NJ (US)

(72) Inventors: Chad Alan Tagge, Sandy, UT (US); Yiping Ma, Layton, UT (US)

(21) Appl. No.: 16/837,767

(22) Filed: Apr. 1, 2020

Related U.S. Application Data
(60) Provisional application No. 62/829,458, filed on Apr. 4, 2019.

(51) Int. Cl. A61M 39/10; A61M 25/06

(52) U.S. Cl. CPC A61M 39/10; A61M 25/0606; A61M 2039/0036; A61M 2039/1077; A61M 2039/1072; A61M 25/0693

(57) ABSTRACT

A catheter assembly may include a catheter adapter, which may include a distal end, a proximal end, and a lumen extending through the distal end and the proximal end of the catheter adapter. The catheter assembly may include a septum disposed within the lumen, a septum actuator fixed within the lumen, and a spring disposed within the lumen distal to the septum. The septum may include a distal end, a proximal end, and a barrier that may divide an interior of the septum into a distal cavity and a proximal cavity. In response to insertion of a medical device into the proximal end of the catheter adapter, the septum may move from a proximal position to a distal position, and the septum actuator may penetrate the septum. The spring may return the septum from the distal position to the proximal position in response to removal of the medical device.

### RELATED APPLICATION

This application claims the benefit of U.S. Application No. 62/829,458, filed Apr. 4, 2019, and entitled MULTI-USE BLOOD CONTROL CATHETER ASSEMBLY, which is incorporated herein in its entirety.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous catheter (“PIVC”). As its name implies, the over-the-needle PIVC may be mounted over an introducer needle having a sharp distal tip. The PIVC and the introducer needle may be assembled so that the distal tip of the introducer needle extends beyond the distal tip of the PIVC with the bevel of the needle facing up away from skin of the patient. The PIVC and introducer needle are generally inserted at a shallow angle through the skin into vasculature of the patient.

In order to verify proper placement of the introducer needle and/or the PIVC in the vein, a user generally confirms that there is “flashback” of blood in a flashback chamber of a PIVC assembly. Once placement of the needle has been confirmed, a user may temporarily occlude flow in the vein and remove the introducer needle, leaving the PIVC in place within the vein. The PIVC may then be used for fluid infusion and/or blood withdrawal or collection.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices and related systems and methods. More particularly, the present disclosure relates to catheter assemblies. In some embodiments, a catheter assembly may include a catheter adapter, which may include a distal end, a proximal end, and a lumen extending through the distal end of the catheter adapter and the proximal end of the catheter adapter.

In some embodiments, the catheter assembly may include a septum, which may be disposed within the lumen. In some embodiments, the septum may include a distal end, a proximal end, and a barrier disposed between the distal end of the septum and the proximal end of the septum. In some embodiments, the barrier may divide an interior of the septum into a distal cavity and a proximal cavity. In some embodiments, the barrier may include a slit. In some embodiments, the septum may be configured to move from a proximal position to a distal position in response to insertion of a medical device into the proximal end of the catheter adapter.

In some embodiments, the catheter assembly may include a septum actuator, which may be fixed within the lumen. In some embodiments, the septum actuator may be configured to penetrate the slit in response to movement of the septum from the proximal position to the distal position.

In some embodiments, the catheter assembly may include a spring, which may be disposed within the lumen distal to the septum. In some embodiments, the spring may be configured to return the septum from the distal position to the proximal position in response to removal of the medical device from the proximal end of the catheter adapter. In some embodiments, the spring may surround the septum actuator.

In some embodiments, the spring and/or the septum may be constructed of an elastomer. For example, the spring and/or the septum may be constructed of silicon or another suitable material. In some embodiments, the spring and the septum may be monolithically formed as a single unit. In some embodiments, the spring may include a coil, which may surround the septum actuator. In some embodiments, the coil may be constructed of metal or another suitable material. In some embodiments, the coil may include a helical shape or multiple waves.

In some embodiments, the septum actuator may include a tubular body. In some embodiments, an outer surface of the tubular body may be smooth. In some embodiments, the tubular body may be generally cylindrical or tapered.

In some embodiments, the septum actuator may include an annular lip extending outwardly from an outer surface of the tubular body. In some embodiments, the annular lip may extend distally from the outer surface of the tubular body. In some embodiments, the annular lip may be disposed at a proximal end of the septum actuator. In some embodiments, the annular lip may include a generally truncated cone shape.

In some embodiments, an inner surface of the septum forming the distal cavity may include one or more protrusions. In some embodiments, the one or more protrusions may be annular. In some embodiments, the annular lip may be configured to pass the one or more protrusions in response to the septum being moved from the proximal position to the distal position. In some embodiments, the annular lip may be configured to contact the one or more protrusions when the septum is moved from the proximal position to the distal position.

In some embodiments, a portion of the inner surface of the septum forming the distal cavity may be smooth. In these and other embodiments, the inner surface of the septum forming the distal cavity may not include the one or more protrusions. In some embodiments, the portion of the inner surface may be distal to the barrier. In some embodiments, the portion of the inner surface may be aligned with or proximal to the annular lip when the septum is disposed in the proximal position. In some embodiments, the annular lip may be spaced apart from the portion of the inner surface of the septum such that the annular lip does not contact the portion of the inner surface when the septum is moved from the proximal position to the distal position. In some embodiments, the portion of the inner surface may be proximate and/or proximal to an opening of the distal end of the septum.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is an upper perspective view of an example catheter system, according to some embodiments;

FIG. 2A is a partial cutaway view of an example catheter assembly, illustrating an example spring and an example septum disposed in a proximal position, according to some embodiments;

FIG. 2B is a cross-sectional view of the catheter assembly of FIG. 2A, illustrating the spring and the septum disposed in the proximal position, according to some embodiments;

FIG. 2C is a cross-sectional view of the catheter assembly of FIG. 2A, illustrating the spring and the septum disposed in a distal position, according to some embodiments;

FIG. 3A is a partial cutaway view of the catheter assembly, illustrating another example spring and the septum disposed in a proximal position, according to some embodiments;

FIG. 3B is a cross-sectional view of the catheter assembly, illustrating the other spring and the septum disposed in the proximal position, according to some embodiments;

FIG. 3C is a cross-sectional view of the catheter assembly, illustrating the other spring and the septum disposed in a distal position, according to some embodiments;

FIG. 4A is a partial cutaway view of the catheter assembly, illustrating another example septum disposed in a proximal position, according to some embodiments;

FIG. 4B is a cross-sectional view of the catheter assembly, illustrating the other septum disposed in a distal position, according to some embodiments;

FIG. 5A is a partial cutaway view of the catheter assembly, illustrating the other septum disposed in a proximal position and an example septum actuator, according to some embodiments;

FIG. 5B is a cross-sectional view of the catheter assembly, illustrating the other septum disposed in the proximal position and the septum actuator, according to some embodiments;

FIG. 5C is a cross-sectional view of the catheter assembly, illustrating the septum other disposed in a distal position and the septum actuator, according to some embodiments; and

FIG. 6 is an upper perspective view of the septum or the other septum, according to some embodiments.

### DESCRIPTION OF EMBODIMENTS

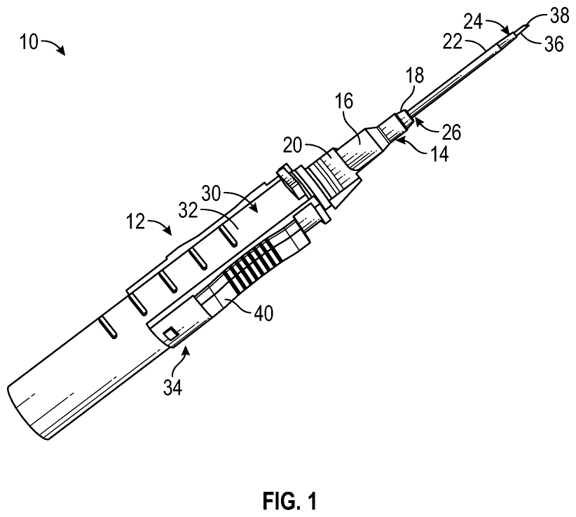

Referring now to FIG. 1, a catheter system 10 is illustrated, according to some embodiments. In some embodiments, the catheter system 10 may include a needle shield device 12 and a catheter assembly 14. In some embodiments, the catheter assembly 14 may include a catheter adapter 16, which may include a distal end 18, a proximal end 20, and a lumen extending through the distal end 18 and the proximal end 20. In some embodiments, the catheter assembly 14 may include a catheter 22, which may include a distal end 24 and a proximal end 26. In some embodiments, the catheter 22 may include a peripheral intravenous catheter (“PIVC”), a midline catheter, a peripherally inserted central catheter (“PICC”), or another suitable catheter. In some embodiments, the proximal end 26 of the catheter 22 may be secured within the catheter adapter 16.

In some embodiments, the catheter system 10 may include one or more features of the safety catheter assembly described in U.S. patent application Ser. No. 15/012,013, filed Feb. 1, 2016, entitled “RELEASABLE CATHETER HUB RETAINER,” which is hereby incorporated by reference in its entirety. In some embodiments, the catheter system 10 may include one or more features of the safety catheter assembly described in U.S. Patent Application Ser. No. 62/808,133, filed Feb. 20, 2019, entitled “IMPROVED COUPLING BETWEEN A TELESCOPING NEEDLE SHIELD AND A CATHETER ADAPTER,” which is hereby incorporated by reference in its entirety.

In some embodiments, the needle shield device 12 may include a needle housing 30, which may be removably coupled to the catheter adapter 16. In some embodiments, the needle housing 30 may include an elongated body 32. In some embodiments, the needle shield device 12 may include a needle assembly 34, which may be slideably coupled to the needle housing 30.

In some embodiments, the needle assembly 34 may include an introducer needle 36, which may include sharp distal tip 38. In some embodiments, the introducer needle 36 may extend through the catheter 22 when the catheter system 10 is in an insertion position ready for insertion into vasculature of a patient, as illustrated, for example, in FIG. 1A. In some embodiments, the needle assembly 34 may include a needle grip 40, which the user may grip and move proximally to withdraw the introducer needle 36 from the vasculature once placement of the catheter 22 within the vasculature is confirmed.

In some embodiments, placement of the catheter 22 within the vasculature may be confirmed via blood flashback. In some embodiments, in response to the introducer needle 36 being inserted into the vasculature of the patient, blood flashback may flow through the sharp distal tip 38 of the introducer needle 36 and out of a distal notch of the introducer needle 36 into a portion of the catheter system 10. For example, the blood flashback may flow through the sharp distal tip 38 and out of the distal notch into a space between an exterior surface of the introducer needle 36 and an interior surface of the catheter 22. In some embodiments, after the blood flashback is observed by the clinician, the introducer needle 36 may be withdrawn, and the needle assembly 34 and the needle housing 30 may be removed from the catheter adapter 16.

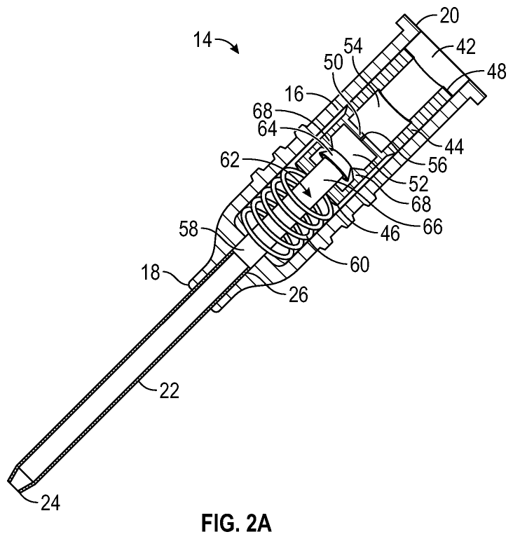

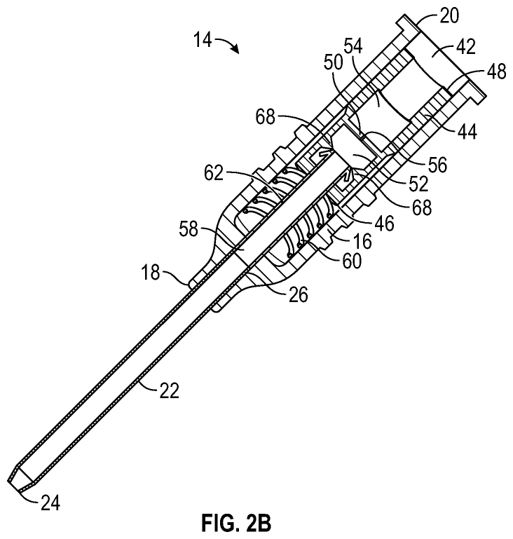

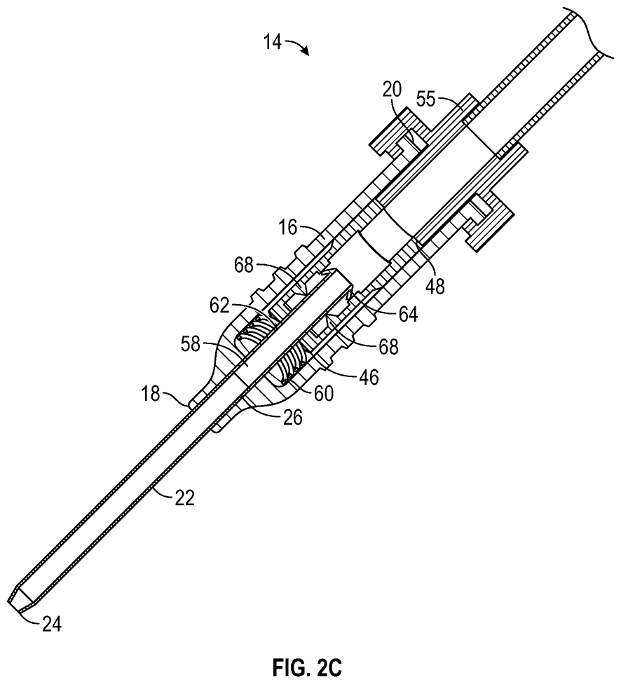

Referring now to FIGS. 2A-2C, in some embodiments, the catheter assembly 14 may include the catheter adapter 16, which may include the distal end 18, the proximal end 20, and a lumen 42 extending through the distal end 18 of the catheter adapter 16 and the proximal end 20 of the catheter adapter 16.

In some embodiments, the catheter assembly 14 may include a septum 44, which may be disposed within the lumen 42. In some embodiments, the septum 44 may include a distal end 46, a proximal end 48, and a barrier 50 disposed between the distal end 46 of the septum 44 and the proximal end 48 of the septum 44. In some embodiments, the barrier 50 may divide an interior of the septum 44 into a distal cavity 52 and a proximal cavity 54. In some embodiments, the barrier 50 may prevent fluid from flowing between the distal cavity 52 and the proximal cavity 54 when the barrier 50 is closed. In some embodiments, the barrier 50 may include a slit 56. In some embodiments, the septum 44 may be configured to move from a proximal position to a distal position in response to insertion of a medical device 55 into the proximal end 20 of the catheter adapter 16.

In some embodiments, the catheter assembly 14 may include a septum actuator 58, which may be fixed within the lumen 42. In some embodiments, the septum actuator 58 may be configured to penetrate the slit 56 in response to movement of the septum 44 from the proximal position to the distal position. In some embodiments, the septum 44 may include a substantially H-shaped cross-section.

In some embodiments, the catheter assembly 14 may include a spring 60, which may be disposed within the lumen 42 distal to the septum 44. In some embodiments, the spring 60 may be configured to return the septum 44 from the distal position to the proximal position in response to removal of the medical device 55 from the proximal end 20 of the catheter adapter 16. Thus, in some embodiments, the spring 60 may facilitate multiple uses of the catheter assembly 14 and the septum 44, including multiple infusions and/or blood withdrawals via multiple connections of the medical device 55 to the catheter adapter 16. In some embodiments, the spring 60 may be compressed in response to movement of the septum 44 to the distal position. In some embodiments, the spring 60 may be decompressed in response to movement of the septum 44 to the proximal position.

In some embodiments, the spring 60 may surround the septum actuator 58, as illustrated, for example, in FIG. 2A. In some embodiments, the spring 60 may include a coil, as illustrated, for example in FIG. 2A, which may surround the septum actuator 58. In some embodiments, the coil may be helical. In some embodiments, the coil may be constructed of metal or another suitable material. In some embodiments, the coil may be constructed of an elastomer. For example, the coil and/or the septum 44 may be constructed of silicon, which may provide improved mechanical properties, or another suitable material. In some embodiments, the coil and the septum 44 may be monolithically formed as a single unit. In some embodiments, a proximal end of the spring 60 may be coupled to the septum 44 and/or a distal end of the spring 60 may be coupled to the catheter adapter 16.

In some embodiments, the spring 60 may include a wave spring, which may be coiled and/or may surround the septum actuator 58. In some embodiments, the wave spring may be made from a coiled wire that includes waves to give the coiled wire a spring effect. In some embodiments, the wave spring may be constructed of metal or another suitable material. In some embodiments, the wave spring may be constructed of an elastomer. For example, the wave spring and/or the septum 44 may be constructed of silicon, which may provide improved mechanical properties, or another suitable material. In some embodiments, the wave spring and the septum 44 may be monolithically formed as a single unit. In some embodiments, a proximal end of the spring 60 may be coupled to the septum 44 and/or a distal end of the spring 60 may be coupled to the catheter adapter 16.

In some embodiments, the septum actuator 58 may include a tubular body 62. In some embodiments, the tubular body 62 may be generally cylindrical or tapered. In some embodiments, the septum actuator 58 may include an annular lip 64 extending outwardly from an outer surface 66 of the tubular body 62. In some embodiments, the annular lip 64 may extend distally from the outer surface 66 of the tubular body 62. In some embodiments, the annular lip 64 may be disposed at a proximal end of the septum actuator 58. In some embodiments, the annular lip 64 may include a generally truncated cone shape, as illustrated, for example, in FIG. 2A. In some embodiments, the annular lip 64 or the generally truncated cone shape may facilitate securement of the septum 44 in the proximal position prior to insertion of the medical device 55 into the proximal end 20 of the catheter adapter 16, providing resistance to distal movement of the septum 44.

In some embodiments, an inner surface of the septum 44 forming the distal cavity 52 may include one or more protrusions 68. In some embodiments, the one or more protrusions 68 may be annular. In some embodiments, the annular lip 64 may be configured to pass the one or more protrusions 68 in response to the septum being moved from the proximal position to the distal position. In some embodiments, the annular lip 64 may be configured to contact the one or more protrusions 68 when the septum 44 is moved from the proximal position to the distal position. In some embodiments, the one or more protrusions 68 may contact the tubular body 62 or the one or more protrusions 68 may not contact the tubular body 62.

In some embodiments, the one or more protrusions 68 and/or the annular lip 64 may provide resistance to the septum 44 returning from the distal position to the proximal position in response to removal of the medical device 55. In further detail, the one or more protrusions 68 may contact or catch on the annular lip 64 when the septum 44 moves towards the proximal position from the distal position and/or the annular lip 64 may contact or catch on the barrier 50 when the septum 44 moves towards the proximal position from the distal position. However, the spring 60 may overcome the resistance to facilitate movement of the septum 44 from the distal position to the proximal position. In this way, in some embodiments, the spring 60 may facilitate multiple uses of the catheter assembly 14 and the septum 44.

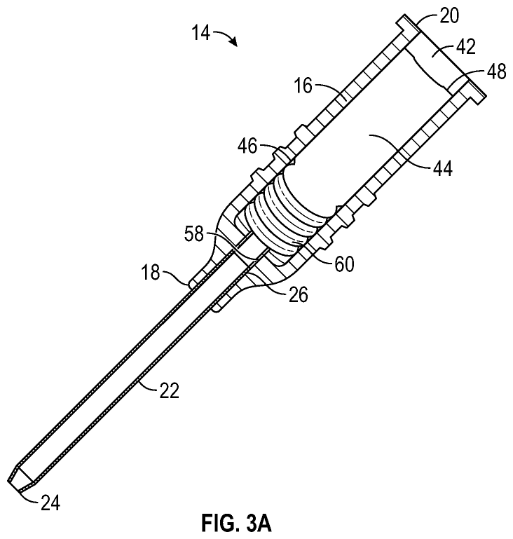

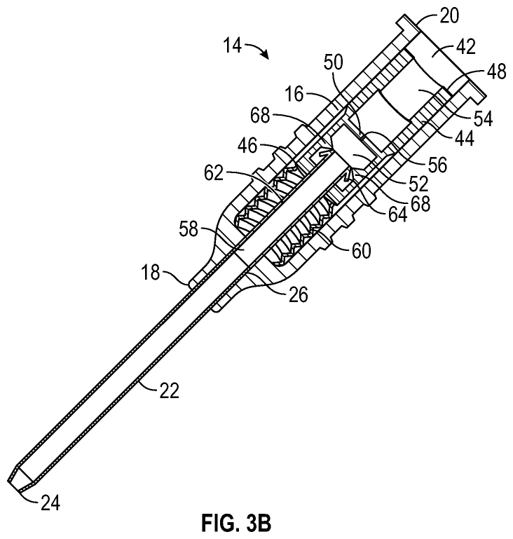

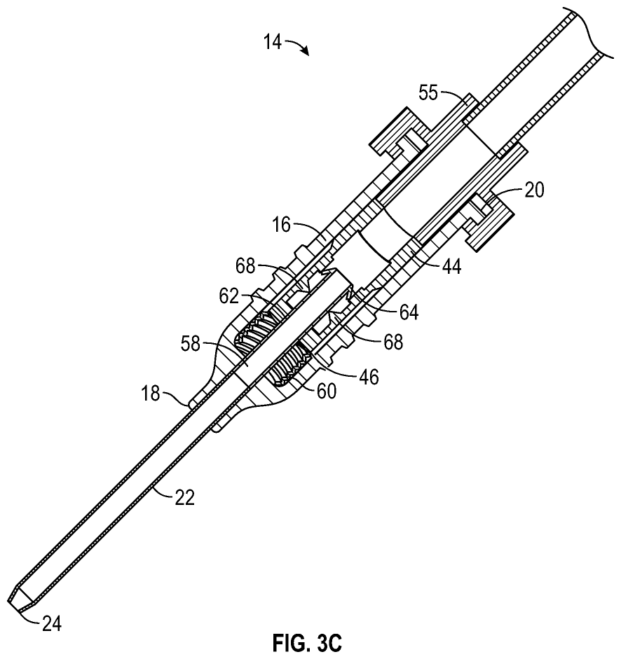

Referring now to FIGS. 3A-3C, in some embodiments, the spring 60 and/or the septum 44 may be constructed of an elastomer. For example, the spring 60 and/or the septum 44 may be constructed of silicon, which may provide improved mechanical properties, or another suitable material. In some embodiments, the spring 60 and the septum 44 may be monolithically formed as a single unit. In some embodiments, the spring 60 may surround the septum actuator 58.

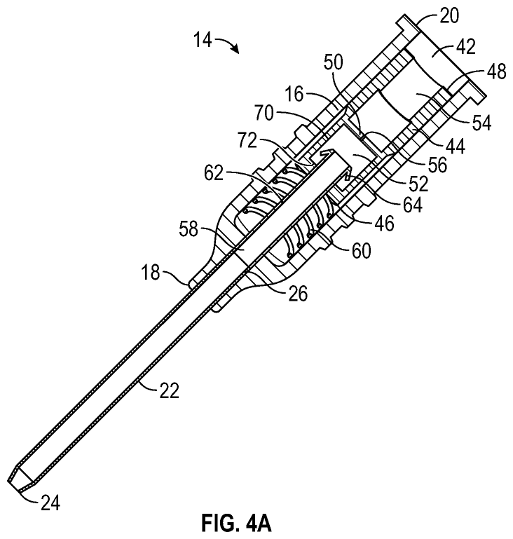

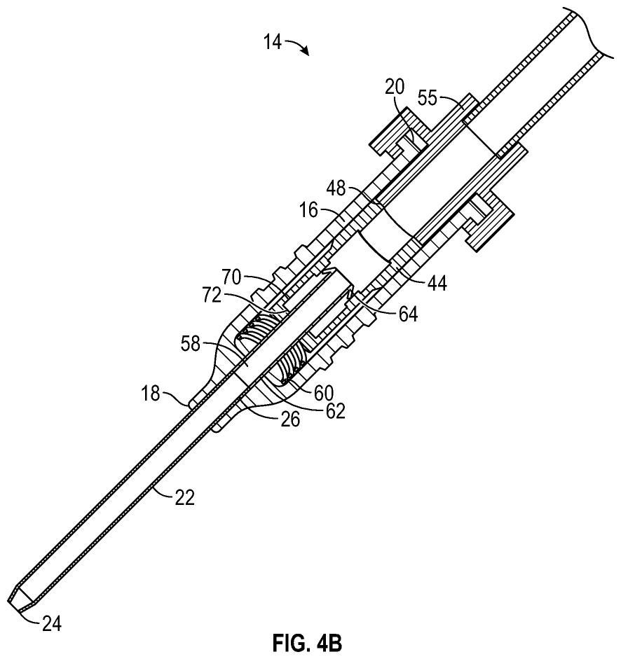

Referring now to FIGS. 4A-4B, in some embodiments, a portion 70 of the inner surface of the septum 44 forming the distal cavity 52 may be smooth. In these and other embodiments, the inner surface of the septum 44 forming the distal cavity 52 may not include the one or more protrusions 68. In some embodiments, the portion 70 of the inner surface may be distal to the barrier 50. In some embodiments, removal of the one or more protrusions 68 may facilitate return of the septum 44 from the proximal position to the distal position and multiple uses of the septum 44.

In some embodiments, the portion 70 of the inner surface may be aligned with or proximal to the annular lip 64 when the septum 44 is disposed in the proximal position. In some embodiments, the annular lip 64 may be spaced apart from the portion 70 of the inner surface of the septum 44 such that the annular lip 64 does not contact the portion 70 of the inner surface when the septum 44 is moved from the proximal position to the distal position. In some embodiments, the portion 70 of the inner surface may be proximate and/or proximal to an opening 72 of the distal end of the septum 44.

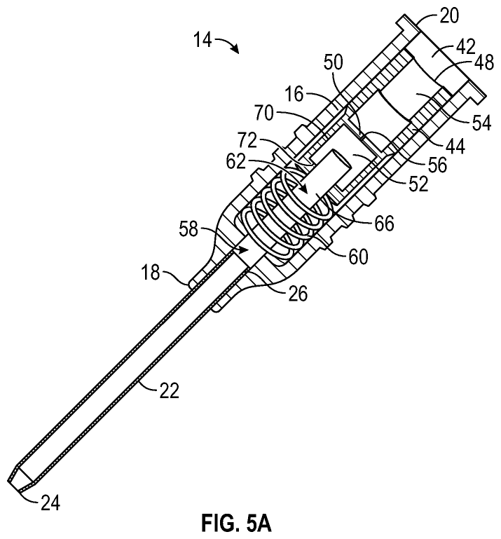

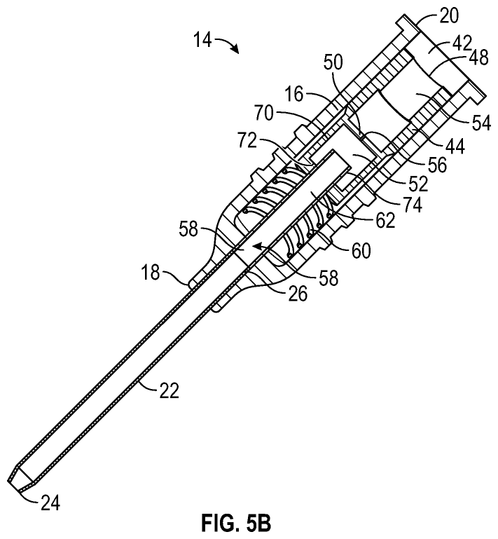

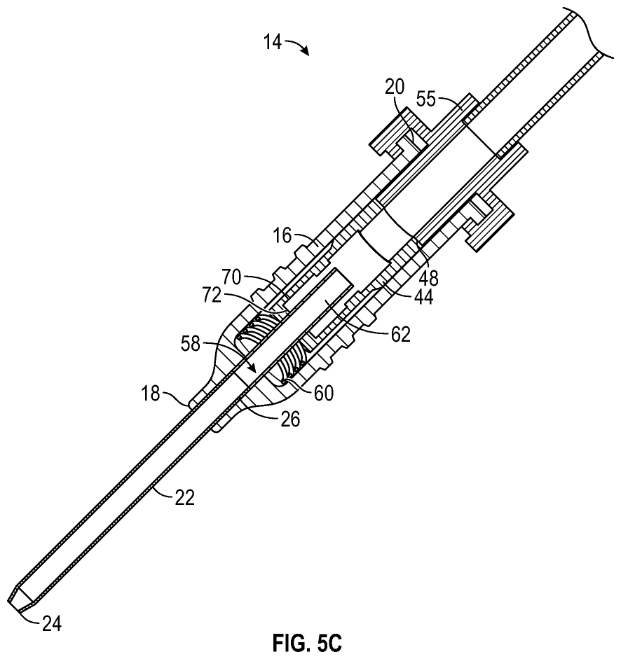

Referring now to FIGS. 5A-5C, in some embodiments, an outer surface of a proximal end 74 of the septum actuator 58 may be smooth and may not include the annular lip 64. In further detail, in some embodiments, an outer surface of the tubular body 62 may be smooth and may not include the annular lip 64. In some embodiments, removal of the annular lip 64 may facilitate return of the septum 44 from the proximal position to the distal position and multiple uses of the septum 44. In some embodiments, an inner surface of the septum 44 forming the distal cavity 52 may include the one or more protrusions 68. In other embodiments, the portion 70 of the inner surface of the septum 44 forming the distal cavity 52 may be smooth.

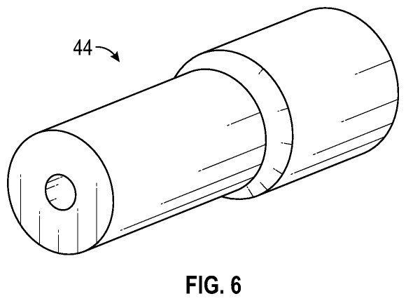

Referring now to FIG. 6, an upper perspective view of the septum 44 is illustrated, according to some embodiments. In some embodiments, an outer surface of the septum 44 may be generally cylindrical.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter assembly, comprising:
a catheter adapter, comprising a distal end, a proximal end, and a lumen extending through the distal end and the proximal end;
a septum disposed within the lumen, wherein the septum comprises a distal end, a proximal end, and a barrier disposed between the distal end of the septum and the proximal end of the septum, wherein the barrier divides an interior of the septum into a distal cavity and a proximal cavity, wherein the barrier comprises a slit, wherein the septum is configured to move from a proximal position to a distal position in response to insertion of a medical device into the proximal end of the catheter adapter;
a septum actuator fixed within the lumen, wherein the septum actuator is configured to penetrate the slit in response to movement of the septum from the proximal position to the distal position; and
a spring disposed within the lumen distal to the septum, wherein the spring is configured to return the septum from the distal position to the proximal position in response to removal of the medical device from the proximal end of the catheter adapter.

2. The catheter assembly of claim 1, wherein the spring surrounds the septum actuator.

3. The catheter assembly of claim 1, wherein the spring and the septum are constructed of an elastomer.

4. The catheter assembly of claim 1, wherein the spring and the septum are constructed of silicon.

5. The catheter assembly of claim 1, wherein the spring and the septum are monolithically formed as a single unit.

6. The catheter assembly of claim 1, wherein the septum actuator comprises a tubular body and an annular lip extending outwardly from an outer surface of the tubular body, wherein the annular lip is disposed at a proximal end of the septum actuator.

7. The catheter assembly of claim 6, wherein an outer surface of the tubular body is smooth.

8. The catheter assembly of claim 6, wherein the annular lip comprises a generally truncated cone shape.

9. The catheter assembly of claim 6, wherein an inner surface of the septum forming the distal cavity comprises a protrusion, wherein the annular lip is configured to pass the protrusion in response to the septum being moved from the proximal position to the distal position.

10. The catheter assembly of claim 1, wherein a portion of an inner surface of the septum forming the distal cavity is smooth, wherein the portion is distal to the barrier, wherein the portion is aligned with or proximal to the annular lip when the septum is disposed in the proximal position.

11. The catheter assembly of claim 1, wherein the spring comprises a coil, wherein the coil surrounds the septum actuator, wherein the coil comprises a helical shape or a plurality of waves.

12. The catheter assembly of claim 11, wherein the coil is constructed of metal.

13. A catheter assembly, comprising:
a catheter adapter, comprising a distal end, a proximal end, and a lumen extending through the distal end and the proximal end;
a septum disposed within the lumen, wherein the septum comprises a distal end, a proximal end, and a barrier disposed between the distal end of the septum and the proximal end of the septum, wherein the barrier divides an interior of the septum into a distal cavity and a proximal cavity, wherein the barrier comprises a slit, wherein the septum is configured to move from a proximal position to a distal position in response to insertion of a medical device into the proximal end of the catheter adapter;
a septum actuator fixed within the lumen, wherein the septum actuator comprises a tubular body and an annular lip extending outwardly from an outer surface of the tubular body, wherein the annular lip is disposed at a proximal end of the septum actuator, wherein the septum actuator is configured to penetrate the slit in response to movement of the septum from the proximal position to the distal position; and
a spring disposed within the lumen distal to the septum, wherein the spring is configured to return the septum from the distal position to the proximal position in response to removal of the medical device from the proximal end of the catheter adapter,
wherein a portion of an inner surface of the septum forming the distal cavity is smooth, wherein the portion is distal to the barrier, wherein the portion is aligned with or proximal to the annular lip when the septum is disposed in the proximal position.

14. The catheter assembly of claim 13, wherein the spring surrounds the septum actuator.

15. The catheter assembly of claim 14, wherein the spring and the septum are monolithically formed as a single unit.

16. The catheter assembly of claim 15, wherein the spring and the septum are constructed of an elastomer.

17. The catheter assembly of claim 16, wherein the annular lip comprises a generally truncated cone shape.

18. The catheter assembly of claim 13, wherein the spring comprises a coil, wherein the coil surrounds the septum actuator.

19. A catheter assembly, comprising:
a catheter adapter, comprising a distal end, a proximal end, and a lumen extending through the distal end and the proximal end;
a septum disposed within the lumen, wherein the septum comprises a distal end, a proximal end, and a barrier disposed between the distal end of the septum and the proximal end of the septum, wherein the barrier divides an interior of the septum into a distal cavity and a proximal cavity, wherein the barrier comprises a slit, wherein the septum is configured to move from a proximal position to a distal position in response to insertion of a medical device into the proximal end of the catheter adapter;
a septum actuator fixed within the lumen, wherein the septum actuator comprises a tubular body and an annular lip extending outwardly from an outer surface of the tubular body, wherein the annular lip is disposed at a proximal end of the septum actuator, wherein the septum actuator is configured to penetrate the slit in response to movement of the septum from the proximal position to the distal position; and
a spring disposed within the lumen distal to the septum, wherein the spring is configured to return the septum from the distal position to the proximal position in response to removal of the medical device from the proximal end of the catheter adapter,
wherein an inner surface of the septum forming the distal cavity comprises a protrusion, wherein the annular lip is configured to pass the protrusion in response to the septum being moved from the proximal position to the distal position.

20. The catheter assembly of claim 19, wherein the spring and the septum are constructed of an elastomer and monolithically formed as a single unit.
